(19) United States
(12) Patent Application Publication
(10) Pub. No.: US 2016/0262944 A1
(43) Pub. Date: Sep. 15, 2016

(54) SILICONE STRIP

(71) Applicant: Kast Silicon Ltd., Bet Shemesh (IL)

(72) Inventors: Asher Shmuelovitch, Tel Aviv (IL); Ishai Kadouri, Tel Aviv (IL)

(21) Appl. No.: 14/642,785

(22) Filed: Mar. 10, 2015

(51) Int. Cl. A61F 13/02; A61L 15/26; A61L 15/42; C09J 7/02

(52) U.S. Cl. CPC A61F 13/0246; C09J 7/0239; A61L 15/26; A61L 15/42; A61L 2400/18

(57) ABSTRACT

A dressing for the treatment of scar tissue. The dressing includes a silicone strip and an adhesive frame to securely attach the silicone strip to an area of scar tissue and where the frame partially covers the silicone strip.

### CROSS-REFERENCE TO RELATED APPLICATIONS

This application claims priority from U.S. Provisional Patent Application: 61/950,855, filed Mar. 11, 2014, which is hereby incorporated in its entirety by reference.

### FIELD OF THE INVENTION

The present invention relates to silicone strips and the treatment of scars.

### BACKGROUND OF THE INVENTION

Silicone is known in the art as a medicinal solution for the treatment of scar tissue. The silicone is typically applied to the scar area via a strip (usually made from silicone gel) and is usually held in place using some form of adhesive strip or bandage. One example of this is shown in FIG. 1, to which reference is now made, which illustrates the bandage described in U.S. Pat. No. 6,284,941. Bandage 10 comprises a silicone strip 20 which is adhered to a piece of skin using adhesive strip 30. Other examples, such as US Patent Publication US2009104252 include silicone strips that are made from an adhesive material and which are adhered to skin without the aid of an adhesive strip.

### SUMMARY OF THE PRESENT INVENTION

There is provided, in accordance with a preferred embodiment of the present invention, a dressing for the treatment of scar tissue. The dressing includes a silicone strip and an adhesive frame to securely attach the silicone strip to an area of scar tissue where the frame partially covers the silicone strip.

Moreover, in accordance with a preferred embodiment of the present invention, the dressing is sized to fit a standard scar size.

Further, in accordance with a preferred embodiment of the present invention, the frame includes an open area to enable oxygen penetration to the silicon strip.

Still further, in accordance with a preferred embodiment of the present invention, the silicone strip is manufactured from liquid silicone rubber.

Additionally, in accordance with a preferred embodiment of the present invention, the adhesive fame is manufactured from medical adhesive tape.

There is provided, in accordance with a preferred embodiment of the present invention, a method for the treatment of scar tissue. The method includes enabling oxygen penetration to the scar tissue via a silicon strip while securely attaching the silicone strip to the scar tissue.

Moreover, in accordance with a preferred embodiment of the present invention, the method includes sizing the silicone strip to fit a standard scar size.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a silicone bandage;

FIGS. 2A and 2B are schematic illustrations of a silicone bandage, constructed and operative in accordance with the present invention;

FIG. 3 is a flowchart of the manufacture process of the silicone bandage of FIGS. 2A and 2B; and

FIGS. 4A and 4B are schematic illustrations of the placement of a silicone strip on an adhesive frame 60 during the manufacture of the silicone bandage of FIGS. 2A and 2B.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicants have realized that aside from the healing capabilities of silicone directly placed over scar tissue, the healing effects of the active ingredient within the silicone may be reinforced by increasing the amount of oxygen available to the affected area of skin, since studies have shown that oxygen significantly improves the healing process.

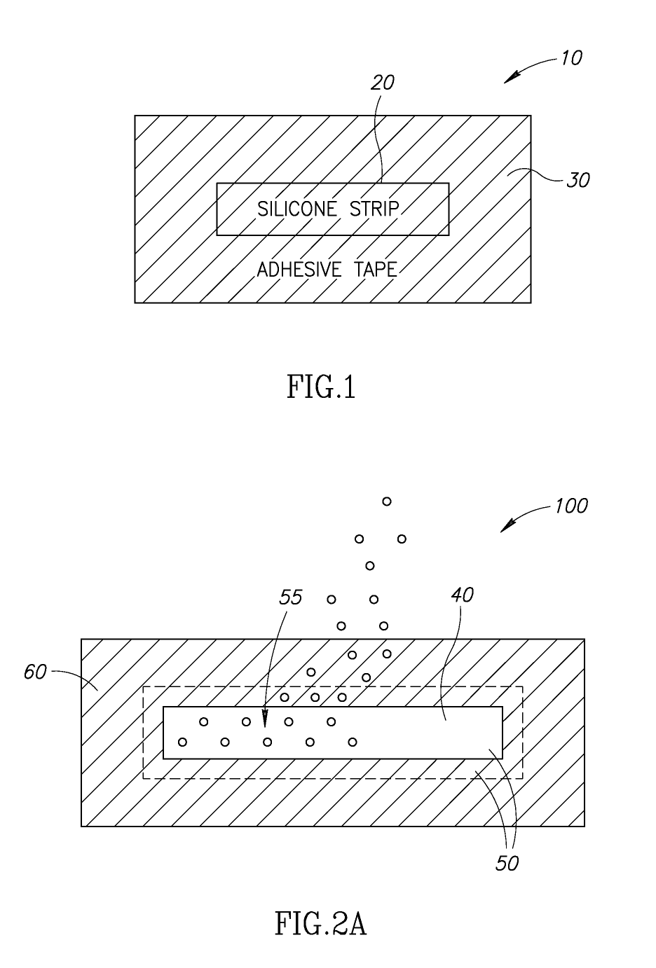

It will be appreciated that although the silicone bandage as illustrated in FIG. 1 may securely adhere a silicone strip to a scarred area of skin, the extra layer of the adhesive strip may limit potential oxygen from penetrating through the silicone strip to the skin, even if manufactured from a breathable material. Applicants have further realized that although the silicone strips that are manufactured from adhesive materials do not have this extra layer and allow oxygen to penetrate, they may not be adhesive enough to securely hold the silicone strip in place.

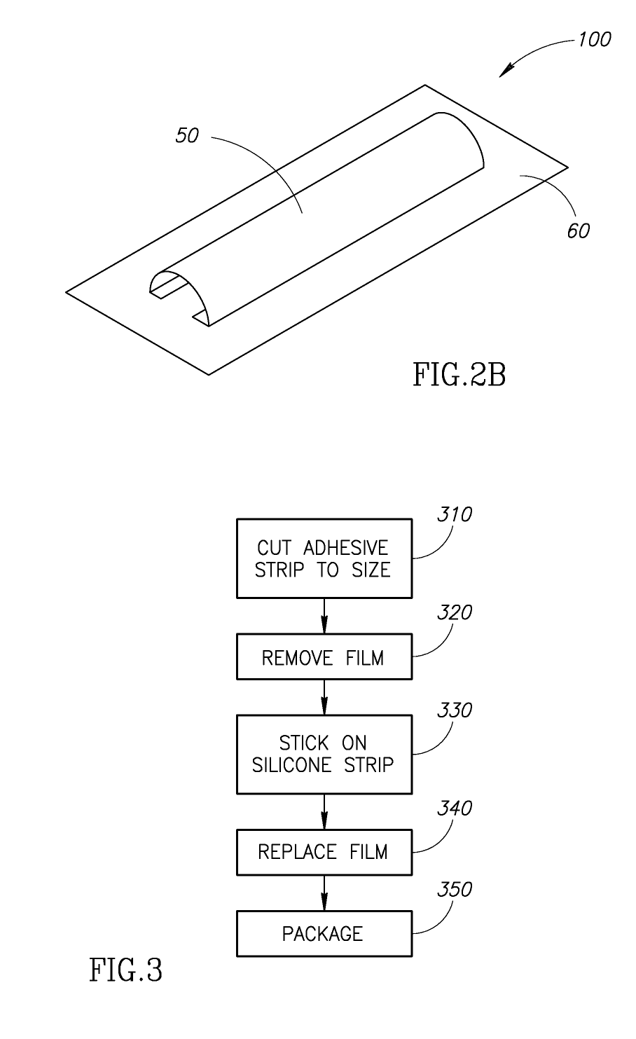

Reference is now made to FIGS. 2A and 2B which illustrate a silicone dressing 100, constructed and operative in accordance with a preferred embodiment of the present invention, which may be adhesively attached over scar tissue. Dressing 100 may comprise a silicone strip 50 which may have one side to be placed over scar tissue whilst its secondary side may be attached to an adhesive frame 60. Adhesive frame 60 may allow silicone strip 50 to be adhered to the pertinent skin area.

It will be appreciated that since adhesive frame 60 may not entirely cover silicone strip 50, additional oxygen may penetrate (shown via arrows 55) via an uncovered area 40 of silicone strip 50.

Silicone strip 50 may be typically manufactured from liquid silicone rubber such as SILBIONE Soft Liquid Silicone Rubbers for Healthcare. Adhesive frame 60 may typically a medical adhesive tape such as Single Coated Nonwoven Medical Silicone Adhesive Tape on Premium Liner manufactured by 3M.

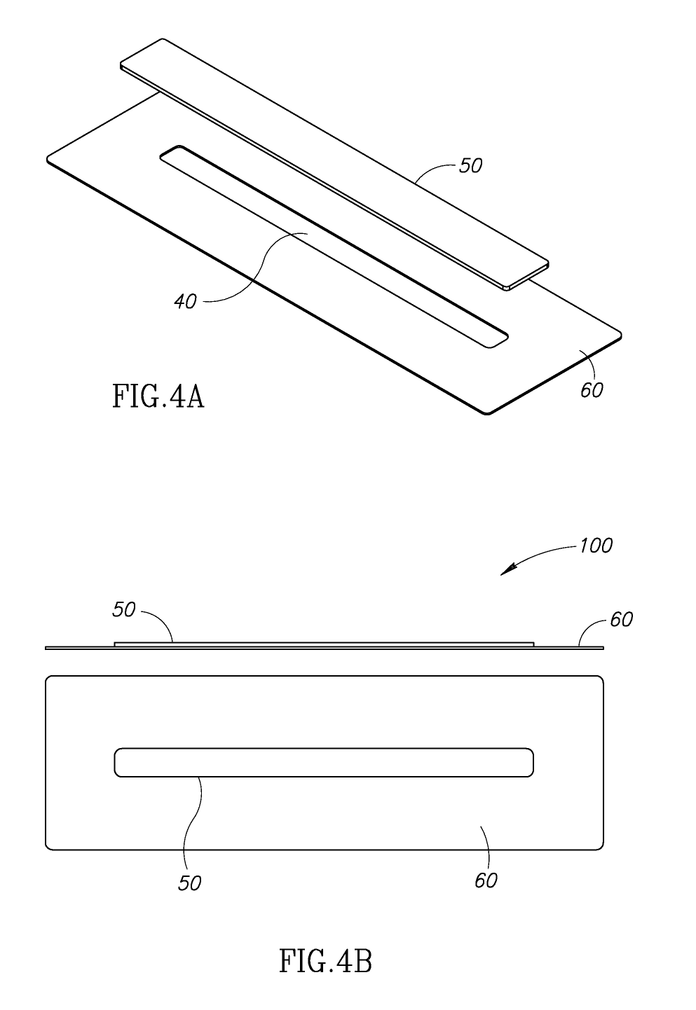

Reference is now made to FIG. 3 which shows a flowchart of the manufacture process of dressing 100. Adhesive frame 60 may be cut by laser from a regular strip of medical adhesive tape (step 310). The regular protective backing of the adhesive tape may then be removed (step 320) and silicone strip 50 may be strategically placed over frame 60 (step 330) as is illustrated in FIG. 4A to which reference is now made. Once silicone strip 50 has been stuck in place, the protective backing that was removed in step 320 may be replaced (step 240) to continue to protect the adhesive area of the adhesive tape. Dressing 100 is now ready to be packaged for later use (step 350). It will be appreciated that dressing 100 may not require a secondary protective backing on its non-adhesive side (over silicone strip 50) to preserve sterility.

Reference is now made to FIG. 4B which illustrates the upper side of silicone dressing 100 (away from the skin). It will be appreciated that due to its thickness, silicone strip 50 may slightly protrude outwards from uncovered area 40. This may also be seen in FIG. 2B.

It will be appreciated that commercially available silicone bandages/dressings may often be sold in bulk sizes to be cut to size according to the size of the scar in question. Such bulk sizes may be cut to size allowing for an adjustment of width but may have a standard length (and vica versa). Applicants have further realized that dressing 100 may be manufactured and sized to comfortably fit over standard scars, such as those as a result of an surgical operation, e.g. an appendicitis operation, a caesarian section, etc. Typical bandage sizes may be approximately 10 cm for a caesarian section and 5 cm for a mastectomy bandage.

Therefore a standardized bandage or dressing may be used to securely adhere a silicone strip to an area of scarred skin, while also allowing for maximum oxygen penetration to potentially accelerate the healing of the scar in question.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A dressing for the treatment of scar tissue, said dressing comprising:
a silicone strip; and
an adhesive frame to securely attach said silicone strip to an area of scar tissue, wherein said frame partially covers said silicone strip.

2. The dressing according to claim 1 and wherein said dressing is sized to fit a standard scar size.

3. The dressing according to claim 1 and wherein said frame comprises an open area to enable oxygen penetration to said silicon strip.

4. The dressing according to claim 1 and wherein said silicone strip is manufactured from liquid silicone rubber.

5. The dressing according to claim 1 and wherein said adhesive fame is manufactured from medical adhesive tape.

6. A method for the treatment of scar tissue, said method comprising enabling oxygen penetration to said scar tissue via a silicon strip while securely attaching said silicone strip to said scar tissue.

7. The method according to claim 6 and also comprising sizing said silicone strip to fit a standard scar size.
